(19) United States
(12) Patent Application Publication
(10) Pub. No.: US 2026/0223840 A1
(43) Pub. Date: Aug. 6, 2026

(54) COMPOSITIONS AND METHODS FOR DISINFECTING REMOVABLE DENTAL APPLIANCES

(71) Applicant: LMA Solutions Inc., Raleigh, NC (US)

(72) Inventors: Nicholas Conley, Raleigh, NC (US); Lynn Muzik, Raleigh, NC (US)

(21) Appl. No.: 19/354,244

(22) Filed: Oct. 9, 2025

Related U.S. Application Data
(63) Continuation of application No. 17/693,298, filed on Mar. 11, 2022, now abandoned.
(60) Provisional application No. 63/160,832, filed on Mar. 14, 2021.

(51) Int. Cl. A01N 37/02; A01N 25/00; A01N 25/12; B08B 3/08

(52) U.S. Cl. CPC A01N 37/02; A01N 25/00; A01N 25/12; B08B 3/08

(57) ABSTRACT

Dental or oral appliance cleansing compositions not used in the mouth are described as having an effervescence system and a surfactant. The cleansing composition can remove a biofilm adherent to the dental or oral appliance; does not contain an artificial dye, does not contain >70 wt. % sodium dodecyl sulfate or sodium lauryl sulfate with respect to total surfactant wt. percent, does not contain sulfonate- or sulfoacetate-containing surfactants, does not contain phosphate- or amine-containing chelating agents, does not contain a bleaching agent, and is able to remove greater than 46% of biofilm from the dental or oral appliance soaked in the cleansing composition in about 15 minutes.

### CROSS REFERENCE TO RELATED APPLICATION

This application claims priority to U.S. Provisional Pat. No. 63/160,832, filed Mar. 14, 2021 entitled Compositions and Methods for Disinfecting Removable Dental Appliances and is hereby incorporated by reference.

### FIELD

The present teachings relate to compositions, methods of producing and use of an effective composition for disinfecting, deodorizing, and removing biofilm from removable dental and oral appliances.

### BACKGROUND

Consumers are becoming increasingly conscious of the ingredients that are used in personal care products and other consumer packaged goods, and for this subset of consumers, purchasing decisions are increasingly driven by the ingredients label. Some consumers have been conditioned to dislike certain ingredients. Companies whose formulations do not include certain ingredients will often list prominently on their own packaging, “Contains no X, Y, or Z,” to condition the consumer to develop an aversion to competitors' products that may contain such ingredients. Another consideration for ingredient-conscious consumers is their preference toward shorter ingredient lists (i.e., a desire for no unnecessary ingredients). Therefore, the use of multi-functional ingredients to reduce the size of the ingredient list is ideal when targeting this consumer segment.

U.S. Pat. No. 8,044,008 to Applicants discloses a dental appliance cleansing composition containing a bleaching agent, a surfactant, an effervescence system, a sequestering agent for alkaline earth metals, a drying agent, a dye, and an effervescent solution to indicate cleansing is complete when a color change occurs.

U.S. Pat. No. 8,937,036 to Applicants discloses a dental appliance cleansing composition which contains a chloramine bleaching agent and an amino acid. The amino acid component dramatically potentiates the disinfecting properties of the chloramine bleaching agent, allowing the latter to be incorporated at lower percentages, which reduces a chlorine-like odor detected by sensitive consumers upon dissolution in water. It is also believed that the amino moiety of the amino-acid forms an N—Cl bond with chlorine from hypochlorous acid (formed upon dissolution of the composition in water) and reduces the concentration of the hypochlorous acid species, which is responsible for the chlorine-like odor.

U.S. Pat. No. 9,205,161 to Applicants is a Continuation-In-Part of U.S. Pat. Nos. 8,937,036 and 8,044,008 which also includes an amino-substituted alkylsulfonic acid to further address the chlorine-like odor and reduce the amount of chloramine bleaching agent while maintaining the disinfecting properties.

U.S. Pat. No. 9,561,161 to Florman discloses a method of cleansing a dental or oral appliance while the appliance is in the wearer's mouth. The need to spray the cleanser formulation in such a way that the cleanser formulation will reside between the appliance and the person's teeth can result in spilling and bleaching of the wearer's clothing due to the composition containing hydrogen peroxide. There is also the risk of ingestion of the cleansing composition, potential drug interactions, upset stomach from the hydrogen peroxide and the detergents, and allergic responses.

EXISTING COMMERCIAL PRODUCTS-Consumers are becoming increasingly conscious of the ingredients that are used in personal care products and other consumer packaged goods, and for this subset of consumers, purchasing decisions are increasingly driven by the ingredients label. The following ingredients, when appearing on the ingredients label, may evoke negative sentiment among consumers due to concerns about irritation, toxicity, or simply a lack of necessity: artificial dyes (i.e. without a biological or natural origin); these are sometimes referred to as “synthetic dyes,” but this is not an accurate classification since dyes of biological origin can be synthesized in a laboratory and are otherwise indistinguishable from their extracted counterparts); sodium dodecyl sulfate (SDS) or sodium lauryl sulfate (SLS) surfactants/detergents; sulfonate- or sulfoacetate-containing detergents; phosphate- or amine-containing chelating agents; and bleaching (oxidizing or reducing) agents.

Several products for cleansing removable dental and oral appliances are commercially available (Table 1), but all possess one or more of these less preferred ingredients on the ingredient label.

TABLE 1

| | | SLS/SDS, | | |
|---|---|---|---|---|
| | | sulfonate- or | | |
| | | sulfoacetate- | | |
| | | containing | Phosphate or amine- | |
| Product | | surfactants/ | containing | Bleaching |
| Name | Artificial Dyes | detergents | chelating agents | agents |
| Applicant's | FD&C Blue | Sodium | Sodium tripoly- | Sodium |
| US 8,044,008 | No. 2 | dodecyl | phosphate | dichloroiso- |
| | | sulfate | | cyanurate |
| Applicant's A | none | Sodium | Sodium tripoly- | Sodium |
| product (US | | dodecyl | phosphate | dichloroiso- |
| 8,937,036 & | | sulfate | | cyanurate |
| US 9,205,161) | | | | |
| Competitor F | FD&C Blue | Sodium lauroyl | Tetraacetylethylene- | Potassium |
| | #1, aluminum | sulfoacetate | diamine | mono- |
| | lake (CI | | | persulfate, |
| | 42090), FD&C | | | * sodium |
| | Blue #2 (CI | | | percarbonate |
| | 73015) | | | |
| Competitor G | Blue 1 | Sodium lauroyl | Tetraacetylethylene- | Potassium |
| | aluminum | sulfoacetate | diamine | mono- |
| | lake, Blue 2, | | | persulfate, |
| | Yellow 5 | | | * Sodium |
| | Aluminum | | | perborate |
| | lake, Yellow 5 | | | |
| Competitor H | Indigo (CI | Sodium lauryl | Tetraacetylethylene- | Potassium |
| | 73015) | sulfate | diamine | peroxy- |
| | | | | monosulfate, |
| | | | | * Sodium |
| | | | | percarbonate |

* This ingredient has been implicated by FDA in serious allergic reactions and at least one death.

Therefore, there remains a need in the art for creating and producing dental and oral appliance cleaners for use outside the mouth that are safe, non-toxic, and environmentally friendly and made with or derived from naturally occurring ingredients.

### SUMMARY

The absence of any dental or oral appliance cleansing product in the marketplace that is (1) free of artificial dyes; does not contain >70 wt. % sodium dodecyl sulfate or sodium lauryl sulfate with respect to total surfactant wt. percent; is free of sulfonate- or sulfoacetate-containing surfactants, phosphate- or amine-containing chelating agents, and bleaching agents; (2) is able to remove greater than 46% of biofilm from the dental or oral appliance soaked in a solution of the cleansing composition in about 15 minutes; (3) claims ability to cause at least a 3-log 10 reduction in bacteria; and (4) has the ability to remove biofilm has created a long felt but unsolved need in the marketplace and serves as evidence of the failure of others.

The present invention meets these needs. An important advantage of this invention is to provide an effective composition for disinfecting, deodorizing, cleansing and removing beverage and food stains and biofilm from removable dental and oral appliances that does not contain any of the following: artificial dyes; >70 wt. % sodium dodecyl sulfate or sodium lauryl sulfate surfactants; sulfonate- or sulfoacetate-containing surfactants; phosphate or amine-containing chelating agents; or bleaching agents. Removable dental and oral appliances include removable braces (clear aligners), retainers, night (bruxism) guards, mouthguards (e.g., for sports), dentures, partials, bridges, and other appliances that are designed to be removable from the mouth by the wearer (e.g., for intermittent cleansing).

In one embodiment, disclosed is a dental or oral appliance cleansing composition having (a) an effervescence system; and (b) a surfactant. The cleansing composition can remove a biofilm adherent to a dental or an oral appliance when the dental or oral appliance is soaked in a solution of the cleansing composition and greater than 46% of biofilm is removed from the dental or oral appliance as measured in a “Biofilm Removal Test” when the dental or oral appliance is soaked in the solution of the cleansing composition for about 15 minutes.

In another embodiment, the claimed cleansing composition does not contain at least one of: Greater than 70 wt. % sodium dodecyl sulfate or sodium lauryl sulfate with respect to total weight of the surfactant; does not contain sulfonate- or sulfoacetate-containing surfactants; does not contain phosphate- or amine-containing chelating agents; does not contain a bleaching agent; and does not contain an artificial dye.

In yet another embodiment, the cleansing composition's effervescence system consists essentially of a water-soluble carboxylic acid and an alkaline base and the number of acid equivalents in the water-soluble carboxylic acid can be present in stoichiometric excess of the number of hydroxide base equivalents in the alkaline base. The water-soluble carboxylic acid can be citric acid. And, the alkaline base can be selected from the group consisting of sodium carbonate, sodium bicarbonate, potassium carbonate, potassium bicarbonate, and combinations thereof. The alkaline base can be sodium bicarbonate.

In another embodiment, the surfactant can be selected from the group consisting of sodium coco sulfate, ammonium lauryl sulfate, potassium lauryl sulfate, sodium lauroyl sarcosinate, alkyl glucosides, cocamidopropyl betaine, and mixtures thereof. The surfactant can be sodium coco sulfate and can be present in the cleansing composition in a range of 0.1 wt. % to 10 wt. % by total weight of the cleansing composition.

In still yet another embodiment, the cleansing composition can further comprise a fragrance, wherein the fragrance is lemon oil extract. The fragrance can be present in the cleansing composition in a range of 0.01 wt. % to 1 wt. % by total weight of the cleansing composition.

In a further embodiment, the cleansing composition can further have at least one dye selected from the group consisting of beta-carotene, curcumin, and coreopsis pigments and combinations thereof. The dye can be beta-carotene.

In a still further embodiment, the cleansing composition can be in tablet form or the cleansing composition can be in powder form.

In another embodiment, the cleansing composition enables a greater than 3-log 10 reduction of one or more the following bacteria in 5 minutes when contacted with water: Prevotella (Bacteroides) melaninogenica, Treponema denticola, Porphyromonas gingivalis, Porphyromonas endodontalis, Prevotella intermedia, Bacteroides loescheii, Enterobacteriaceae, Tannerella forsythensis (Bacteroides forsythus), Centipeda periodontii, Eikenella corrodens, Fusobacterium nucleatum, and/or Fusobacterium periodonticum. In yet another embodiment, a solution of the cleansing composition disinfects the dental or oral appliance when the dental or oral appliance is soaked in the solution of the cleansing composition and wherein the solution of the cleansing composition deodorizes the dental on oral appliance when the dental or oral appliance is soaked in the solution of the cleansing composition.

In still yet another embodiment, the effervescence system can be present in the cleansing composition in a range of 90 wt. % to 99 wt. % by total weight or in a range of 50 wt. % to 99 wt. % of the cleansing composition, and the surfactant can be present in the cleansing composition in a range of 1 wt. % to 5 wt. % by total weight of the cleansing composition, and the cleansing composition can further have a fragrance that can be present in the cleansing composition in a range of 0.01 wt. % to 1 wt. % by total weight of the cleansing composition. In another embodiment, the cleansing composition can consist essentially of the effervescence system, the surfactant, and the fragrance or the cleansing composition can consist essentially of the effervescence system, the surfactant, the fragrance, and a dye.

In another embodiment, disclosed is a method of disinfecting a removable dental or oral appliance or a method of deodorizing a removable dental or oral appliance in which an aqueous solution of the cleansing composition is formed; placing said removable dental or oral appliance in the aqueous solution; and removing said removable dental or oral appliance from said aqueous solution after a period of time has elapsed between about 5 minutes and about 24 hours; wherein exposure of said removable dental or oral appliance for 5 minutes in said aqueous solution results in greater than a 3-log 10 reduction in bacterial levels and/or wherein exposure of said removable dental or oral appliance for about 20-45 minutes in said aqueous solution results in an odorless dental or oral appliance.

In another embodiment, disclosed is a dental or oral appliance cleansing composition having (a) an effervescence system; and (b) surfactant; wherein the cleansing composition does not contain at least one of: Greater than 70 wt. % sodium dodecyl sulfate (SDS) or sodium lauryl sulfate (SLS) with respect to total weight of the surfactant; wherein the cleansing composition does not contain sulfonate- or sulfoacetate-containing surfactants; wherein the cleansing composition does not contain phosphate- or amine-containing chelating agents; and/or wherein the cleansing composition does not contain a bleaching agent.

In yet another embodiment, the cleansing composition's effervescence system can be present in the cleansing composition in a range of 90 wt. % to 99 wt. % by total weight of the cleansing composition, the surfactant can be present in the cleansing composition in a range of 1 wt. % to 5 wt. % by total weight of the cleansing composition, and the cleansing composition further comprises a fragrance that can be present in the cleansing composition in a range of 0.01 wt. % to 1 wt. % by total weight of the cleansing composition. The cleansing composition can consist essentially of the effervescence system, the surfactant, and the fragrance.

In still yet another embodiment, the cleansing composition removes a biofilm adherent to a dental or an oral appliance when the dental or oral appliance is soaked in a solution of the cleansing composition; and wherein greater than 46% of biofilm can be removed from the dental or oral appliance as measured in a “Biofilm Removal Test” when the dental or oral appliance is soaked in the solution of the cleansing composition for about 15 minutes.

INCORPORATION BY REFERENCE—All publications, references, patents, and patent applications mentioned in this document are herein incorporated by reference to the same extent as if each individual publication, reference, patent, or patent application was specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

Reference will now be made in detail to certain claims of the invention, examples of which are illustrated in the accompanying drawings. While the invention will be described in conjunction with the enumerated claims, it will be understood that they are not intended to limit those claims. On the contrary, the invention is intended to cover all alternatives, modifications, and equivalents, which can be included within the scope of the invention as defined by the claims.

### BRIEF DESCRIPTION OF THE DRAWINGS

FIG. 1 is a graph illustrating the claimed cleansing composition's efficacy versus marketed products in reduction in biofilm (%) adherence to a removeable dental appliance, as measured by spectrophotometric quantitation.

FIG. 2 are photos of matched, cleaned vs. uncleaned results for a removeable dental appliance using the disclosed cleansing composition versus marketed products at four time points.

### DETAILED DESCRIPTION OF VARIOUS EMBODIMENTS

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting.

Disclosed herein are embodiments for producing water-soluble cleansing compositions for removeable dental and oral appliances comprising safe, non-toxic, environmentally friendly ingredients, made with or derived from naturally occurring raw materials, and methods for using the compositions to clean and remove beverage and food stains, biofilms as well as disinfecting, deodorizing the appliance(s).

DEFINITIONS—Unless defined otherwise, all technical and scientific terms used herein have the same meaning as commonly understood by one of ordinary skill in the art to which this invention belongs. As used herein, the recited terms have the following meanings. The following definitions are included to provide a clear and consistent understanding of the specification and claims. Any methods and materials similar or equivalent to those described herein can be used in the practice or testing of the disclosed invention, as it will be understood that modifications and variations are encompassed within the spirit and scope of the instant disclosure.

References in the specification to “one embodiment,” “an embodiment,” “another embodiment,” and the like, indicate that the described embodiment can include a particular aspect, feature, structure, moiety, or characteristic, but every embodiment may not necessarily include the particular aspect, feature, structure, moiety, or characteristic. Moreover, such phrases are not necessarily referring to the same embodiment. Further, when a particular aspect, feature, structure, moiety, or characteristic is described in connection with an embodiment, it is submitted that it is within the knowledge of one of ordinary skill in the art to effect or connect such aspect, feature, structure, moiety, or characteristic in connection with other embodiments whether or not explicitly described.

It is further noted that the claims may be drafted to exclude an optional element. As such, this statement is intended to serve as antecedent basis for the use of exclusive terminology, such as “solely,” “only,” “other than”, and the like, in connection with any element described herein, and/or the recitation of claim elements or use of “negative” limitations.

The term “and/or” means any one of the items, any combination of the items, or all of the items with which this term is associated. The phrases “one or more” and “at least one” when read in context of its usage are readily understood by one of skill in the art. For example, the phrase can mean one, two, three, four, five, six, ten, 100, or any upper limit approximately 10, 100, or 1000 times higher than a recited lower limit.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range as if each numerical value and sub-range is explicitly recited. As a non-limiting example, each range discussed herein can be readily broken down into a lower third, middle third and upper third, as well as nested ranges within a larger range, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges

For example, a range of “about 1% to about 5%” or “about 0.1% to 5%” should be interpreted to include not just about 1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The term “about” as used herein can allow for a degree of variability in a value or range, for example, within 10%, within 5%, or within 1% of a stated value or of a stated limit of a range. For example, “about 50 percent” can in some embodiments carry a variation from 45 to 55 percent. In yet another example, “about 10.0 wt. %” can be between 9.5 wt. % and 10.5 wt. %.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, element, the composition, or the embodiment. The term about can also modify the end-points of a recited range as discuss above in this paragraph.

In this document, the terms “a,” “an,” or “the” are used to include one or more than one unless the context clearly dictates otherwise. Thus, for example, a reference to “a component” includes a plurality of such components, so that a component Z includes a plurality of components Z. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; Information that is relevant to a section heading may occur within or outside of that particular section.

In the methods or processes described herein, the steps can be carried out in any order without departing from the principles of the invention, except when a temporal or operational sequence is explicitly recited.

Furthermore, specified steps can be carried out concurrently unless explicit claim language recites that they be carried out separately. For example, a claimed step of doing A and a claimed step of doing B can be conducted simultaneously within a single operation, and the resulting process will fall within the literal scope of the claimed process.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The terms “biofilm,” “bio-film,” “bio film,” and plural forms thereof are used interchangeably and broadly include a deposit of cellular microorganisms adherent to surfaces including, but not limited to, teeth and removable dental and oral appliances. Biofilm, which consists of a slimy extracellular matrix composed of a polymeric aggregate of polysaccharides, proteins, lipids and nucleic acids, allows bacteria and other microorganisms like fungi to stick to one another and to dental and oral appliances.

Biofilms contribute to bacterial contamination and dental plaque accumulation on dental and oral appliances resulting in odors, discoloration and staining. The close contact to teeth and gums of dental and oral appliances covered by biofilm(s) contributes to dental plaque accumulation on teeth and gums, which may initiate oral bacterial infection, halitosis, tooth decay, gingivitis, periodontal disease, as well as tooth loss, discoloration and staining.

Biofilms are also implicated in an estimated 80% of a wide variety of microbial infections in the body including bacterial vaginosis, catheter, urinary tract, and middle-ear infections, and coating contact lenses. Although less common, biofilms are also associated with more lethal processes including, but not limited to, lung infections in cystic fibrosis patients, endocarditis, cutaneous wounds, and permanent indwelling devices such as intervertebral disc, heart valve(s), and joint prostheses.

The term “effervescence” as used herein broadly describes the release of carbon dioxide gas from an aqueous solution. There can be at least two effervescent components, as dry reagents (e.g., granules or powders with no more than about ambient moisture). At least one of the effervescent components comprises a base and at least one of the effervescent components comprises an acid. Non-limiting examples of suitable base effervescent components can include, but are not limited to, salts such as sodium carbonate, sodium bicarbonate, potassium carbonate, potassium bicarbonate, lithium carbonate, lithium bicarbonate, and any other metal carbonate or metal bicarbonate compound that when combined with the acid effervescent component in an aqueous solution reacts to form a gaseous product, i.e., the release of carbon dioxide. Non-limiting examples of suitable acid effervescent components can include, but are not limited to, citric acid (fruit juice), acetic acid (vinegar), carbonic acid, tartaric acid, itaconic acid, and any other organic acid that reacts with the base effervescent component.

The phrase “effervescence system;” as used herein can describe the powder or tablet form of at least two effervescent components, as substantially moisture-free reagents comprising at least a base and an acid. The effervescent system may also include excipients, such as desiccants or inert chemical coatings on the effervescent components, designed to prevent premature effervescence occurring due to environmental humidity. The composition of the effervescence system when dissolved in an aqueous solution releases carbon dioxide, fizzes and may foam as the components are uniformly dispersed and dissolved in the solution.

The phrase “naturally occurring” as used herein can refer to an ingredient that occurs in nature or is substantially derived from a naturally occurring ingredient. An example, though not to be limiting, is lemon oil. Lemons grow in nature and lemon oil is extracted from lemons. An example of an ingredient derived from a naturally occurring ingredient is sodium coco sulfate which is derived from coconut oil extracted from coconuts that grow in nature. In contrast, “artificial” ingredients are fabricated principally with “man-made” precursors not found in nature. An example of an artificial ingredient is the artificial sweetener acesulfame K. The term “synthetic” does not necessarily imply “artificial” or “non-naturally occurring” since synthetic compounds can be prepared in the laboratory that are chemically indistinguishable from those that occur in nature.

The term “surfactant” as used herein broadly describes agents that reduce the surface tension between two liquids, a gas and a liquid or a liquid and a solid. The surfactant can act as at least one of a foaming agent, dispersant, detergent, emulsifier and wetting agent. Examples of surfactants include but are not limited to anionic surfactants including sodium coco sulfate, sodium dodecyl sulfate (SDS), sodium lauryl sulfate (SLS) as well as sodium and potassium derivatives of sulfonates, phosphates, and carboxylates.

The disclosed cleansing composition uses sodium coco sulfate derived from coconut oil. The coconut oil is an amalgam of most saturated fatty acids (C8-C18). The saturated C12-C18 subset of these fatty acids used in the manufacture of sodium coco sulfate include lauric acid (about 50%), myristic acid (about 16%), palmitic acid (about 8%), and stearic acid (about 2%) (https://www.hebebotanicals.co.nz/sodium-coco sulfate-another-synthetic-detergent/). Upon sulfonation, these fatty acids create a distribution of sulfonated products with different chain lengths. During manufacturing of sodium lauryl sulfate, coconut oil is processed to remove most of the non-12 carbon fatty acids before the fatty acids are sulfated.

Because lauric acid is about 66% of the total fatty acid content in the fraction of coconut oil used to make sodium coco sulfate (50%/[50%+16%+8%+2%]=65.8%) and about 3.95 wt. % of sodium coco sulfate is used in Applicant's B and C compositions, the total SLS content in Applicant's B and C compositions is therefore 0.0395×0.658=0.0260 or 2.6% SLS; this is consistent with Applicant's A Product (U.S. Pat. Nos. 8,937,036 and 9,205,161).

The calculation of “at least 3 log reduction” as used herein describes the log₁₀ (bacteria titer before treatment with cleansing agent)−log₁₀ (bacteria titer after treatment with cleansing agent)≥3-log₁₀. Data from studies reported herein include measurements of both the log reduction of bacteria in suspension and biofilm removal. In contrast, competitor products may only measure log reduction of bacteria in suspension, which is not representative of the biofilm in which bacteria exist when colonizing dental and oral appliances

Chemical products for cleansing removable dental appliances are classified by the FDA as Class I Medical Devices that are exempt from the premarket notification requirement, except when such products make claims about removing pathogenic microorganisms, in which case they are classified as “prescription dental cleansers” and require 510(k) clearance (see: https://www.accessdata.fda.gov/scripts/cdrh/cfdocs/cfpcd/315.cfm?GMPPart=872). However, when such products make only claims about removing odor-causing bacteria, they are classified as “over-the-counter (OTC) dental cleansers.” Typically, these OTC products claim 3-log₁₀ (i.e. 99.9%) reduction of odor-causing bacteria, which is the standard for a disinfectant claim. By narrowing the claim to “odor-causing bacteria,” the product is not regulated as a prescription dental cleanser, and no 501 (k) clearance is required.

To address the long-felt but unsolved need for dental and oral appliance cleansing products that appeal to ingredient-conscious consumers, alternative compositions have been developed that retain efficacy (≥3-log 10 reduction in 5 minutes) while avoiding ingredients that some consumers may perceive as unnecessary or unhealthy.

To that end, an alternative to artificial dyes is the use of no dye. Additionally, there are several plant-based dyes (e.g., naturally occurring or derived from plants found in nature) that may be acceptable to ingredient-conscious consumers. For example, suitable plant-based orange dyes may include at least one orange dye selected from the group consisting of beta-carotene, curcumin, and coreopsis pigments and combinations thereof. The weight percentage of dye in the claimed composition can be zero or a value of about 0.0010, 0.0011, 0.0012, 0.0013, 0.0014, 0.0015, 0.0016, 0.0017, 0.0018, 0.0019, 0.002, 0.003, 0.004, 0.005, 0.006, 0.007, 0.008, 0.009, 0.01, 0.0125, 0.015, 0.0175, 0.018, 0.019, 0.02, 0.0225, 0.025, 0.0275, 0.028, 0.029, 0.03, 0.0325, 0.035, 0.0375, 0.038, 0.039, and 0.04 percent. The actual percentage of dye will depend on the molar absorptivity (i.e., the extinction coefficient) of the dye. Excess dye percentages, e.g., about 0.075 to about 0.1 percent or more dye could stain dental and oral appliances during the cleansing process.

Unlike with dyes, the omission of a surfactant is not ideal for dental and oral appliance cleansing products. However, there are a number of surfactants that are much more acceptable to ingredient-conscious consumers than sodium dodecyl sulfate (SDS); sodium lauryl sulfate (SLS); and sulfonate- or sulfoacetate-containing surfactants. These include, but are not limited to, for example, sodium coco sulfate, ammonium lauryl sulfate, potassium lauryl sulfate, sodium lauroyl sarcosinate, alkyl glucosides (e.g., decyl or lauryl glucoside), and cocamidopropyl betaine. The weight percentage of surfactant can be about 1.0, 1.25, 1.5, 1.75, 1.8, 1.9, 2.0, 2.25, 2.5, 2.75, 2.8, 2.9, 3.0, 3.25, 3.5, 3.75, 3.8, 3.9, 4.0, 5.0, 6.0, 7.0, 8.0, 9.0 and 10.0 percent.

A common misconception among persons of ordinary skill in the art of dental and oral appliance cleansing products is that phosphate- or amine-containing chelating agents and/or bleaching agents are required to achieve 3-log₁₀ reduction of bacteria for dental and oral appliance cleansing tablets. An important aspect of the present invention is the recognition that water-soluble carboxylic acids, like citric acid, can serve 3 simultaneous roles: (1) contributing acid equivalents to the effervescent system, (2) acting as a chelator to improve the surfactant activity, and (3) serving as the active ingredient that enables 3-log 10 reduction in bacteria (in lieu of a bleaching agent). Not only does citric acid serve three purposes, thereby reducing the length of the ingredient list, but it eliminates the need for a harsh bleaching agent to meet the disinfectant claim. Bleaching agents (oxidizing agents) can damage plastics and metals used in removable dental and oral appliances and are unattractive to ingredient-conscious consumers.

To maximize effervescence, sodium bicarbonate is preferred over sodium carbonate, as the former consumes less acid equivalents from the acid component and produces more CO₂ effervescence on a weight basis.

In one non-limiting example embodiment, the cleansing composition comprises the effervescence system (e.g., citric acid and sodium bicarbonate), the surfactant (e.g., sodium coco sulfate), and the fragrance (e.g., lemon oil) wherein the effervescence system is present in the cleansing composition in a range of 50 wt. % to 99 wt. % by total weight of the cleansing composition, the surfactant is present in the cleansing composition in a range of 1 wt. % to 10 wt. % by total weight of the cleansing composition, and the fragrance is present in the cleansing composition in a range of 0.01 wt. % to 1 wt. % by total weight of the cleansing composition. Alternatively, the effervescence system can be present in the cleansing composition in a range of 60 wt. % to 99 wt. % by total weight of the cleansing composition, or 70 wt. % to 99 wt. % by total weight of the cleansing composition, or 80 wt. % to 99 wt. % by total weight of the cleansing composition, or 90 wt. % to 99 wt. % by total weight of the cleansing composition, or 95 wt. % to 99 wt. % by total weight of the cleansing composition. Alternatively, the surfactant can be present in the cleansing composition in a range of 1 wt. % to 5 wt. % by total weight of the cleansing composition, or 3 wt. % to 5 wt. % by total weight of the cleansing composition. Optionally, wherein the cleansing composition can include a dye (e.g., an orange dye such as beta-carotene) wherein the dye is present in the cleansing composition in a range of 0.001 wt. % to 0.04 wt. % by total weight of the cleansing composition. The cleansing composition may consist essentially of the effervescence system, the surfactant, and the fragrance in any of the wt. % ranges above. The cleansing composition may consist essentially of the effervescence system, the surfactant, the fragrance, and the dye in any of the wt. % ranges above. The cleansing composition is not used in the mouth.

In one non-limiting example embodiment, when a dental or oral appliance is soaked in a solution of the cleansing composition for about 15 minutes, greater than 46% of biofilm is removed from the dental or oral appliance as measured in a “Biofilm Removal Test” described below. In another non-limiting example embodiment, when a dental or oral appliance is soaked in a solution of the cleansing composition for about 30 minutes, greater than 62% of biofilm is removed from the dental or oral appliance as measured in a “Biofilm Removal Test” described below.

The method of cleansing a removable dental or oral appliance outside the mouth can offer both a passive cleansing process and eliminate direct oral exposure to potentially toxic, non-naturally-occurring chemicals. Additionally, ingredient-conscious consumers are averse to potentially ingesting needless chemicals when cleansing a dental or oral appliance in the mouth, which carries the potential of an adverse impact to the microbiomes of both oral and gut sites. Moreover, with ingestion, there also exists the possibility of side effects or drug interactions that would not normally be a consideration for 510(k)-exempt Class I Medical Devices.

### EXAMPLES

### Example 1

### Dental Appliance Cleansing Composition with Natural Fragrance and Dye

The purpose of this formulation was to demonstrate the log reduction achievable (Example 2) from a prototype formulation of the current innovation. Table 2 provides a cleansing composition of the prototype formulation. Approximately 10.2 g of the composition was prepared, mixed to homogeneity, and aliquoted into four vials at 2.55 g of the powder per vial (lot #040719). The contents of one vial was dissolved in 80 mL of warm water (~45° C.). Effervescence with foaming at the surface was observed, along with an accompanying change in the color of the solution from colorless to golden yellow.

TABLE 2

| | | Percent | | Other |
|---|---|---|---|---|
| Ingredient | Manufacturer | (%) | Key Ingredient | Excipients |
| Citric acid, | Transene | 58.82 | Citric acid | None |
| anhydrous, USP | (Oakland, CA) | | | |
| Sodium carbonate, | Transene | 11.76 | Sodium carbonate | None |
| anhydrous, USP | (Oakland, CA) | | | |
| Sodium coco | Wholesale | 1.96 | Sodium coco | None |
| sulfate | Supplies Plus | | sulfate | |
| | (Independence, | | | |
| | OH) | | | |
| Lemon powder | BioFinest | 27.45 | Lemon powder | None |
| | (Lindon, UT) | | | |

### Example 2

### Log Reduction Testing of the Composition in Example 1

The test sample was prepared by adding one vial of the test sample (composition of Example 1) to 78 mL of phosphate buffer solution (PBS) that had been pre-warmed to approximately 45° C. The sample buffer mixture was then inoculated with 2 mL of Escherichia coli culture to obtain a final volume of 80 mL. Escherichia coli (ATCC #11229), a member of the family Enterobacteriaceae, was used for this study.

At the time points of 5, 15, and 30 minutes, 1.0 mL from the inoculated test sample was taken and placed into 9.0 ml of 100 mM of Tris buffer solution (i.e., a 1:10 dilution). Additional 1:10 serial dilutions were prepared using Tris buffer solution to achieve 1:100 and 1:1000 dilutions.

One milliliter from each dilution was plated in duplicate using tryptic soy agar containing lecithin and polysorbate 80 as the growth medium for Escherichia coli. The plates were incubated at 35-37° C. for a minimum of 48 hours. The same procedure was repeated for the PBS control. After the incubation period, colonies on all plates were counted to determine the number of microorganisms remaining at each time point. This experiment was conducted by Bioscreen (Torrance, CA), an accredited independent testing laboratory.

Results are shown in Table 3. The test sample exhibited antibacterial activity against E. coli at all three time points—5 minutes, 15 minutes, and 30 minutes—producing log reduction results of 3.7, 4.8, and 4.8, respectively.

TABLE 3
Escherichia coli ATCC# 11229

| | | CONCENTRATION OF | | | | | |
|---|---|---|---|---|---|---|---|
| | | ORGANISM | | | | | |
| EXPOSURE | | (CFU/mL) | | % REDUCTION | | LOG REDUCTION | |
| TIME | | CONTROL | PRODUCT | CONTROL | PRODUCT | CONTROL | PRODUCT |
| Initial | | 6.2E5 | 6.2E5 | N/A | N/A | N/A | N/A |
| 5 | min. | 6.0E5 | 130 | 3.2 | 99.9 | 0.0 | 3.7 |
| 15 | min. | 6.7E5 | 10 | −8.1 | 99.9 | 0.0 | 4.8 |
| 30 | min. | 5.1E5 | <10 | 17.7 | >99.9 | 0.1 | 4.8 |

### Example 3

### Dental Appliance Cleansing Composition with Natural Fragrance and Dye

Table 4 provides a cleansing composition of the current innovation (“Applicant's B” formula). Approximately 7 kg of the composition was prepared, mixed to homogeneity, and tableted as part of lot #090420CN (ABS Corporation, Omaha, NE). The approximate tablet weight was 2.5 g. The tablet, which had a characteristic lemon oil fragrance, was dissolved in 80 mL of warm water (~45° C.). Effervescence with foaming at the surface was observed, along with an accompanying change in the color of the solution from colorless to golden yellow. Disintegration of the tablet occurred in about 2 minutes, and complete tablet dissolution occurred by about 5 minutes.

TABLE 4

| | | Percent | | Other |
|---|---|---|---|---|
| Ingredient | Manufacturer | (%) | Key Ingredient | Excipients |
| Citrocoat N | Jungbunzlauer | 75.81 | Citric acid | sodium citrate |
| | (Basel, | | | |
| | Switzerland) | | | |
| Sodium | Arm & | 19.59 | Sodium | none |
| bicarbonate, | Hammer | | bicarbonate | |
| anhydrous | (Ewing, NJ) | | | |
| Sodium coco | Colonial | 3.95 | Sodium coco | none |
| sulfate | Chemical | | sulfate | |
| | (Pittsburg, TN) | | | |
| Decolorized | Lebermuth | 0.49 | Lemon oil | none |
| lemon oil, 5x | (Scott Bend, | | | |
| | IN) | | | |
| Beta-Carotene | ZMC-USA | 0.16 | Beta-carotene | gum acacia, |
| Powder (1%) | (Spring, TX) | | | maltodextrin, |
| CWS (cold- | | | | vegetable oil, |
| water soluble) | | | | ascorbyl |
| | | | | palmitate, DL |
| | | | | alpha |
| | | | | tocopherols, |
| | | | | and silicone |
| | | | | dioxide |

### Example 4

### Dental Appliance Cleansing Composition Absent Dye

The purpose of this formulation was to determine if the cleansing composition's efficacy, absent the use of dye, differed when compared to Example 3. The composition of the cleansing formula (“Applicant's C” formula) is presented in Table 5. Approximately 7 kg of the composition was prepared, mixed to homogeneity, and tableted as part of lot #090420CL (ABS Corporation, Omaha, NE). The approximate tablet weight was 2.5 g. The tablet, which had a characteristic lemon oil fragrance, was dissolved in 80 mL of warm water (~45° C.). Effervescence with foaming at the surface was observed. Disintegration of the tablet occurred in about 2 minutes, and complete tablet dissolution occurred by about 5 minutes.

TABLE 5

| | | Percent | | Other |
|---|---|---|---|---|
| Ingredient | Manufacturer | (%) | Key Ingredient | Excipients |
| Citrocoat N | Jungbunzlauer | 75.93 | Citric acid | sodium |
| | (Basel, | | (98.5%) | citrate |
| | Switzerland) | | | (1.5%) |
| Sodium bicarbonate, | Arm & | 19.62 | Sodium | none |
| anhydrous | Hammer | | bicarbonate | |
| | (Ewing, NJ) | | | |
| Sodium coco sulfate | Colonial | 3.96 | Sodium coco | none |
| | Chemical | | sulfate | |
| | (Pittsburg, TN) | | | |
| Decolorized lemon oil, | Lebermuth | 0.49 | Lemon oil | none |
| 5x | (Scott Bend, | | | |
| | IN) | | | |

### Example 5

### Comparative Biofilm Removal Studies of the Compositions in Example 3 and Example 4, Benchmarked Against Leading Competitors' Products (Test Articles) Using the “Biofilm Removal Test.”

### Study Design

### The “Biofilm Removal Test”

The method used to assess the effectiveness of a composition at removing biofilm adherent to a dental or an oral appliance is the “Biofilm Removal Test.”

Test Article details are provided below, copied exactly as they appeared on the manufacture's packaging:

TABLE 6

| Test Article | Lot number | Expiry Date |
|---|---|---|
| Applicant's A product (US | 09491 | April 2021 |
| 8,937,036 & US 9,205,161) | | |
| Applicant's B formula | 090420CN | N/A |
| (Example 3) | | |
| Applicant's C formula | 090420CL | N/A |
| (Example 4) | | |
| Competitor D | 20183B | 2022 Jul. 1 |
| Competitor E | none | December 2021 |
| Competitor F | 0205101 | June 2023 |
| Competitor G | 20G195RG C | 2023 Jun. 30 |

Surplus Invisalign SmartTrack® aligners were obtained from a dentist's office.

An emulsion of EleCare Jr. (Abbott Laboratories, Chicago, IL, USA) was prepared by vigorously shaking 259.2 g of powder in 1200 mL of distilled water, and the emulsion was inoculated with human saliva (~10 mL). Forty Invisalign SmartTrack® aligners were statically incubated in this culture for 14 days at 37° C. in an Erlenmeyer flask, with passage into fresh media being performed every 3-4 days. During each passage, ~2% of the previously incubated growth media was carried over to inoculate the fresh media.

After incubation for 14 days, the 40 aligners were rinsed thoroughly in distilled water and then stained by soaking for 30 minutes in a solution of 40 GUM Red-Cote Plaque Disclosing tablets (Sunstar, River Forest, IL, PN #8U-CMXH-Z3IQ) crushed by mortar and pestle and dissolved in 1000 ml of distilled water. After soaking in dye solution, the aligners were gently rinsed with distilled water.

After staining, all 40 aligners were cut in half, perpendicular to the plane formed by their “U” shape, to give two “J”-shaped pieces per aligner that were approximately mirror images of one another. Both halves of each aligner were kept in a separate labeled container (i.e. 1a, 1b; 2a, 2b; . . . ; 40a, 40b) so that the paired nature between the “a” series and “b” series of each half-aligner was preserved.

Each of thirty-two “a” series half-aligners were stripped of the red dye and biofilm by sonicating in 50 mL of 10% (w/v) sodium dodecyl sulfate (SDS) solution at 50° C. for 5 minutes, and the absorption of a 1.5 mL-aliquot of each solution was measured at 537 nm, the wavelength of maximum of absorption of the D&C Red No. 28 dye. These absorption measurements provided an indication of the amount of biofilm on each “a” series half-aligner, and this biofilm burden should approximately match the biofilm burden on the corresponding “b” series half-aligner in pairs 1-32.

The corresponding 32 half-aligners of the right, “b series” were split into 8 groups of 4 half-aligners each, and each group was subjected to one of eight soak-based cleansing methods (Applicant's A product, Applicant's B formula (Example 3), Applicant's C formula (Example 4), Competitor D, Competitor E, Competitor F, Competitor G, and a water-only control) at 4 time points (15 minutes, 30 minutes, 8 hours, and 24 hours). For each time point (i.e., each half-aligner) a separate cleansing solution was prepared using 70 mL of 50° C. water.

After cleansing each right, “b” series half-aligner in the Test Articles for the prescribed time, the half-aligners were gently rinsed with distilled water and re-stained by soaking for 30 minutes in a solution of 8 GUM Red-Cote Plaque Disclosing tablets crushed by mortar and pestle and dissolved in 200 mL of distilled water. After soaking in dye solution, the half-aligners were gently rinsed with distilled water. Each right, “b” series half-aligner was stripped of the red dye and biofilm by sonicating in 50 ml of 10% (w/V) sodium dodecyl sulfate solution at 50° C. for 5 minutes, and the absorption of a 1.5 mL-aliquot of each solution was measured at 537 nm, the wavelength of maximum of absorption of the D&C Red No. 28 dye. These absorption measurements give an indication of the amount of biofilm that remained on each right, “b” series half-aligner after cleansing.

For each half-aligner pair, the amount of biofilm removed was calculated using the following equation:

$$\% biofilm removed = [ 1 - ( {ABS}_{b - series , n} / {ABS}_{a - series , n} ) ] \times 100 , \qquad [ Equation 1 ]$$

where ABS_{b-series,n} is the absorption value, measured at 537 nm, of the stripped solution for the cleaned right, “b” series half-aligner in the nth pair and ABS_{a-series,n} is the absorption value, measured at 537 nm, of the stripped solution for the uncleaned left, “a” series half-aligner in the nth pair.

For photography, 7 of the 8 remaining sets of half-aligner pairs (i.e., 7 in the left “a” series and 7 in the right, “b” series) were used. To demonstrate uniform biofilm coating and staining, each set of 7 half-aligner pairs were photographed next to one another at t=0. The left half of each pair (“a” series) was not subject to any cleansing, and served as the unchanging reference in each photo. The right half (“b” series) was subjected to one of seven soak-based cleansing methods (Applicant's A product), Applicant's B formula, Applicant's C formula, Competitor D, Competitor E, Competitor F, and Competitor G; the water-only control was not photographed) at 4 time points (15 minutes, 30 minutes, 8 hours, and 24 hours). Cleansing solutions were prepared using each Test Article dissolved in 70 mL of 50° C. water. At the 15-minute time point, each “b” series half-aligner was removed from the cleansing solution, gently rinsed with distilled water and re-stained by soaking for 30 minutes in a solution of 8 GUM Red-Cote Plaque Disclosing tablets crushed by mortar and pestle and dissolved in 200 ml of distilled water. After soaking in dye solution, the half-aligners were gently rinsed with distilled water. The excess water was removed and the cleaned “b” series half-aligner (right half) was photographed next to the corresponding uncleaned “a” series half-aligner (left half). After photography, each “b” series aligner was cleaned in the appropriate cleansing solution (freshly prepared) for an additional amount of time to get to the next time point (i.e., an additional 15 minutes to achieve 30 minutes of total cleansing for the second time point), and the re-straining, rinsing, photographing, and cleansing process was repeated until photographs at all 4 times points in all 7 cleansing solutions were obtained.

### SUMMARY OF RESULTS FROM EXAMPLES 3-5

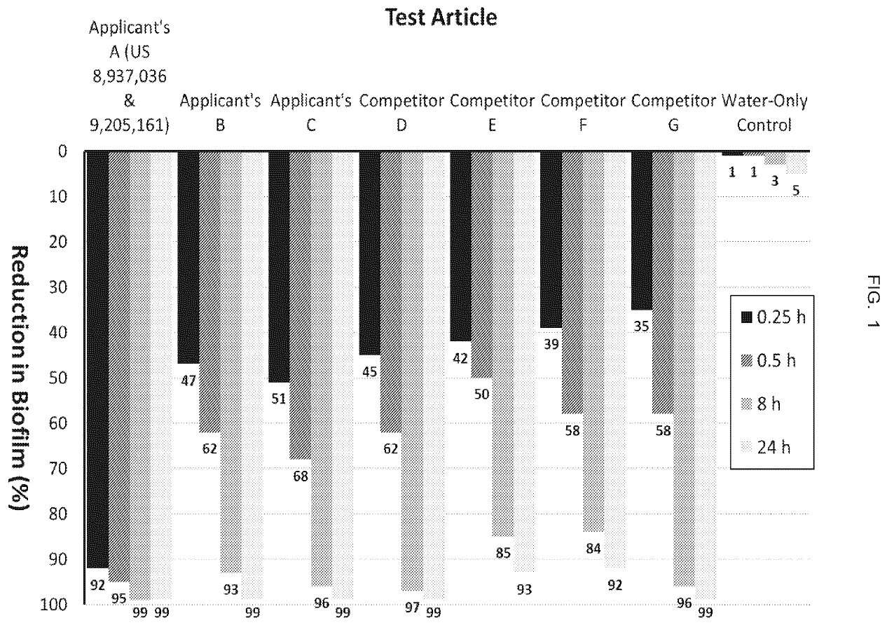

The percentage of biofilm removed at each time point was quantified according to Equation 1 and is plotted for each Test Article in FIG. 1. Applicant's B formula) and Applicant's C formula showed comparable biofilm-removing ability to leading competitors' products. Competitor E and Competitor F slightly underperformed other products in biofilm removal at all time points. Applicant's A product was superior to all products tested at 0.25 hours, 0.5 hours, and 8 hour time points. The amount of biofilm removed by the water soak (control) was not appreciable.

FIG. 1 graphically illustrates quantitative reduction in biofilm for various cleansing formulas by Product Name: Applicant's A product (U.S. Pat. No. 8,937,036 & U.S. Pat. No. 9,205,161), Applicant's B formula), Applicant's C formula, Competitor D, Competitor E, Competitor F, Competitor G, and a water-only control) at 4 time points (15 minutes, 30 minutes, 8 hours, and 24 hours).

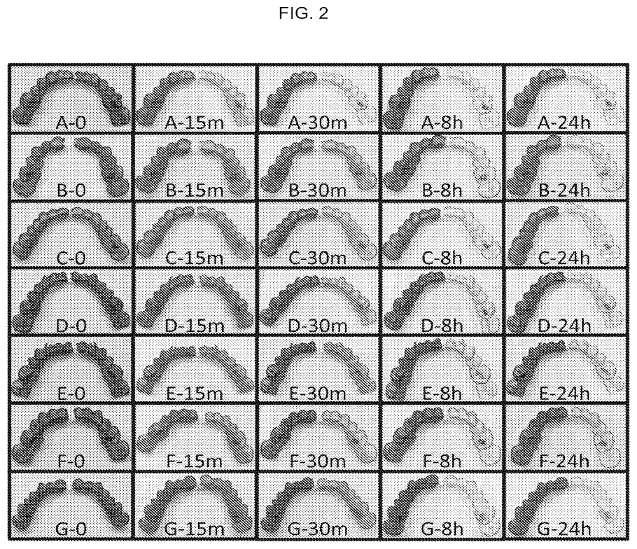

Although the biofilm removal is most accurately quantified using a spectrophotometer, photography of a set of half-aligners in which the “b” series (right half) was subjected to each cleansing solution was also performed, as shown in FIG. 2. For example, it is evident that the right half-aligner in panels A-15m (Applicant's A product, 15 minutes time point) and A-30m (Applicant's A product, 30 min time point) have much less dye stain than right “b” series half-aligners cleaned in the other cleansing solutions at the same time points. In addition, it can be seen in panels E-24h (Competitor E, 24 hour time point) and F-24 (Competitor F, 24 hour time point) that some red stain remains on the right “b” series half-aligners at the 24 hour cleansing time point, in contrast to the photos of right “b” series half-aligners cleaned in other cleansing solutions. Collectively, the photographs of the dye stain in FIG. 2 are consistent with the trends observed in the spectrophotometric quantitation of percentage of biofilm removed shown in FIG. 1.

While the principles of this invention have been described in connection with specific embodiments, it should be understood clearly that these descriptions are made only by way of example and are not intended to limit the scope of the invention. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalence.

1. A dental or oral appliance cleansing composition having a total number of non-water ingredients of four or less, the composition comprising: (a) an effervescence system; and (b) a surfactant; wherein the cleansing composition removes a biofilm adherent to a dental or an oral appliance when the dental or oral appliance is soaked in a solution of the cleansing composition; and wherein greater than 46% of biofilm is removed from the dental or oral appliance when the dental or oral appliance is soaked in the solution of the cleansing composition for about 15 minutes.

2. The cleansing composition according to claim 1, wherein the effervescence system consists essentially of a water-soluble carboxylic acid and an alkaline base.

3. The cleansing composition according to claim 2, wherein the number of acid equivalents in the water-soluble carboxylic acid is present in stoichiometric excess of the number of hydroxide base equivalents in the alkaline base.

4. The cleansing composition according to claim 2, wherein the water-soluble carboxylic acid is citric acid.

5. The cleansing composition according to claim 2, wherein the alkaline base is selected from the group consisting of sodium bicarbonate, sodium carbonate, potassium carbonate, potassium bicarbonate, and combinations thereof.

6. The cleansing composition according to claim 1, wherein the effervescence system is present in the cleansing composition in a range of 50 wt. % to 99 wt. % by total weight of the cleansing composition.

7. The cleansing composition according to claim 1, wherein the surfactant is selected from the group consisting of sodium coco sulfate, ammonium lauryl sulfate, potassium lauryl sulfate, sodium lauroyl sarcosinate, alkyl glucosides, cocamidopropyl betaine, and mixtures thereof.

8. The cleansing composition according to claim 1, the composition further comprising a fragrance.

9. The cleansing composition according to claim 1, further comprising at least one dye selected from the group consisting of beta-carotene, curcumin, and coreopsis pigments and combinations thereof.

10-11. (canceled)

12. The cleansing composition according to claim 1, wherein the composition provides a greater than 3-log₁₀ reduction of one or more the following bacteria in 5 minutes when contacted with water: Prevotella (Bacteroides) melaninogenica, Treponema denticola, Porphyromonas gingivalis, Porphyromonas endodontalis, Prevotella intermedia, Bacteroides loescheii, Enterobacteriaceae, Tannerella forsythensis (Bacteroides forsythus), Centipeda periodontii, Eikenella corrodens, Fusobacterium nudeatum, and/or Fusobacterium periodonticum.

13-14. (canceled)

15. A method of disinfecting a removable dental or oral appliance, the method comprising:
forming an aqueous solution of the cleansing composition according to claim 1;
placing said removable dental or oral appliance in said aqueous solution; and
removing said removable dental or oral appliance from said aqueous solution after a period of time has elapsed between about 5 minutes and about 24 hours;
wherein exposure of said removable dental or oral appliance for 5 minutes in said aqueous solution results in greater than a 3-log₁₀ reduction in bacterial levels.

16. (canceled)

17. A dental or oral appliance cleansing composition having a total number of non-water ingredients of four or less, the composition comprising: (a) an effervescence system; and (b) a surfactant; wherein the cleansing composition does not contain greater than 70 wt. % sodium dodecyl sulfate or sodium lauryl sulfate with respect to total weight of the surfactant, wherein the cleansing composition does not contain sulfonate- or sulfoacetate-containing surfactants, wherein the cleansing composition does not contain phosphate- or amine-containing chelating agents, and wherein the cleansing composition does not contain a bleaching agent.

18. The cleansing composition according to claim 17, wherein: the effervescence system is present in the cleansing composition in a range of 90 wt. % to 99 wt. % by total weight of the cleansing composition, the surfactant is present in the cleansing composition in a range of 1 wt. % to 5 wt. % by total weight of the cleansing composition, and the cleansing composition further comprises a fragrance that is present in the cleansing composition in a range of 0.01 wt. % to 1 wt. % by total weight of the cleansing composition.

19. (canceled)

20. The cleansing composition according to claim 18, wherein the cleansing composition removes a biofilm adherent to a dental or an oral appliance when the dental or oral appliance is soaked in a solution of the cleansing composition; and wherein greater than 46% of biofilm is removed from the dental or oral appliance when the dental or oral appliance is soaked in the solution of the cleansing composition for about 15 minutes.

21. The cleansing composition according to claim 1, wherein the composition is free of artificial dye.

22. The cleansing composition according to claim 1, wherein the composition is free of sodium dodecyl sulfate.

23. The cleansing composition of claim 1, wherein the composition is free of sulfonate- or sulfoacetate-containing detergents.

24. The cleansing composition of claim 1, wherein the composition is free of phosphate- or amine-containing chelating agents.

25. The cleansing composition of claim 1, wherein the composition is free of bleaching agents, oxidizing agents, reducing agents, or a combination thereof.

26. The cleansing composition of claim 1, wherein the composition is free of artificial dye, free of sodium dodecyl sulfate, free of sodium lauryl sulfate, free of sulfonate- or sulfoacetate-containing detergents, free of phosphate- or amine-containing chelating agents, and free of bleaching agents, oxidizing agents, reducing agents, or a combination thereof.
